(19) United States
(12) Patent Application Publication
(10) Pub. No.: US 2015/0126800 A1
(43) Pub. Date: May 7, 2015

(54) SYSTEM AND METHOD FOR MANAGING RADIATION TREATMENT MACHINE

(71) Applicant: Infinitt Healthcare, Co., LTD., Seoul (KR)

(72) Inventors: Bo Ram Kang, Seoul (KR); Moo Hyun Park, Seoul (KR); Hyeon Ji, Seoul (KR)

(73) Assignee: Infinitt Healthcare, Co., LTD.

(21) Appl. No.: 14/596,010

(22) Filed: Jan. 13, 2015

Related U.S. Application Data
(63) Continuation of application No. PCT/KR2012/005668, filed on Jul. 16, 2012.

(30) Foreign Application Priority Data
Jul. 13, 2012 (KR) 10-2012-0076588

(51) Int. Cl. A61N 5/10

(52) U.S. Cl. CPC A61N 5/1075
USPC 600/1

(57) ABSTRACT

A management system and method for managing a radiation treatment machine are disclosed herein. The system includes an information acquisition unit, an amount-of-use database, a treatment plan database, and a management unit. The information acquisition unit acquires information about the amount of use of the radiation treatment machine and information about a treatment plan. The amount-of-use database stores the information about the amount of use of the radiation treatment machine. The treatment plan database stores the time at which treatment is to be performed and information about the amount of use of the radiation treatment machine. The management unit checks whether the cumulative amount of use of the radiation treatment machine to exceed a predetermined reference value when the treatment is to be performed.

### CROSS-REFERENCE TO RELATED APPLICATION

This application is a continuation of PCT/KR2012/005668 filed on Jul. 16, 2012, which claims priority to Korean Application No. 10-2012-0076588 filed on Jul. 13, 2012, which application is incorporated herein by reference.

### TECHNICAL FIELD

The present invention relates to the management of a radiation treatment machine and, more particularly, to a system and method for managing an inspection period based on the amount of use of a radiation treatment machine in order to prevent the failure or erroneous operation of a machine that performs treatment using radiation.

### BACKGROUND ART

Radiation treatment is a treatment method for destructing or reducing cancer cells or malignant tumor cells using high-energy radiation.

When radiation is emitted to the human body, an electrolytic dissociation phenomenon occurs while the radiation is passing through the human body. This causes chemical denaturalization in the nucleic acid or cell membrane essential for the proliferation and existence of cells, thereby destructing tumor cells. In this case, normal cells and cancer cells exhibit different responses to radiation. The normal cells recover from damage over time after radiation treatment, whereas the cancer cells suffer from insufficient recovery. Accordingly, a method of effectively eliminating cancer cells while reducing damage to normal cells through the divided emissions of radiation is employed.

Radiation treatment affects surrounding normal tissue as well as cancer cells because it gives damage to the cancer cells using high energy radiation. For this reason, a side effect attributable to the treatment may occur. In particular, if the trajectory of a radiation beam different from the planned trajectory of a radiation beam is formed, a side effect may be further increased because radiation that should be transferred to malignant tumor cells is transferred to normal tissue. Accordingly, it is important to prevent the failure and erroneous operation of a radiation treatment machine.

FIG. 1 schematically illustrates a system for managing a radiation treatment machine.

Referring to FIG. 1, when the failure or erroneous operation of a radiation treatment machine 110 is detected, an administrator 120 who manages the radiation treatment machine 110 requests an administration engineer 130 to perform a repair or an inspection. When a repair or inspection request is received from the administrator 120 or a regular inspection date has been reached, the administration engineer 130 inspects the radiation treatment machine 110.

In the conventional management of the radiation treatment machine 110, measures are taken according to a predetermined schedule or after the occurrence of a problem, as illustrated in FIG. 1, but an inspection is not conducted based on the amount of use of the radiation treatment machine 110. This makes it difficult to prevent the failure and erroneous operation of a radiation treatment machine and manage an aged radiation treatment machine.

As an example of the conventional radiation treatment management technology, Korean Patent Application Publication No. 2009-0119241 discloses an apparatus and method for managing radiation therapy. This patent application publication discloses a technology in which information about a patient is received from an electronic medical record (EMR) system and an order communication system (OCS), information about the treatment of the patient is provided to and stored in the EMR and the OCS, one or more medical images are received from a picture archiving and communication system (PACS) or a medical imaging apparatus, the information about the patient and the medical images of the patient are provided to a radiation treatment planning (RTP) system so that radiation treatment plan data is prepared, information about the treatment of the patient based on information about a treatment plan is received and stored, and the information about the treatment is sent and stored in the EMR. In accordance with this conventional technology, the radiation treatment management and control system can exchange information with a plurality of medical systems and apparatuses/equipments related to radiation treatment while operating in conjunction with them, but a problem still remains in that the effective management of the use of a radiation treatment machine cannot be achieved even when the conventional technology is employed.

### SUMMARY OF THE DISCLOSURE

Accordingly, the present invention has been made to solve the above problems occurring in the prior art, and an object of the present invention is to provide an apparatus and method for managing an inspection period based on the amount of use of a radiation treatment machine (or a radiation treatment apparatus).

More specifically, an object of the present invention is to provide a management system and method for acquiring information about the amount of use of a radiation treatment machine and sending a report that the radiation treatment machine needs an inspection if the cumulative amount of use of the radiation treatment machine has exceeded a reference value.

Another object of the present invention is to provide a management system and method for acquiring information about a treatment plan and sending a report that the treatment plan needs to be changed or an inspection of the machine is needed if the cumulative amount of use of a radiation treatment machine will exceed a reference value when treatment is to be performed.

Still another object of the present invention is to provide a management system and method for acquiring information about the result of treatment, determining whether the radiation treatment machine is normal or not by analyzing the results of the treatment of patients who have been exposed to a radiation treatment machine, and sending a report that abnormality has occurred in the radiation treatment machine if, as a result of the determination, it is determined that the abnormality is present in the radiation treatment machine.

In accordance with an aspect of the present invention, there is provided a management system for managing a radiation treatment machine, including a storage system and a processor. The processor may include an information acquisition unit configured to acquire information about an amount of use of the radiation treatment machine and information about a treatment plan. The storage device may include an amount-of-use database configured to store the information about the amount of use of the radiation treatment machine; a treatment plan database configured to store a time at which treatment is to be performed and information about an amount of use of the radiation treatment machine when the treatment is to be performed based on the information about the treatment plan. The processor may include also a management unit configured to check whether a cumulative amount of use of the radiation treatment machine will exceed a predetermined reference value when the treatment is to be performed, based on the information about the amount of use of the radiation treatment machine, which is stored in the amount-of-use database, and the time at which the treatment is to be performed and the information about the amount of use of the radiation treatment machine when the treatment is to be performed, which are stored in the treatment plan database, in order to prevent the failure and erroneous operation of the radiation treatment machine.

The information about the amount of use of the radiation treatment machine may include at least one of the amount of radiation of the radiation treatment machine, the number of times that radiation has been emitted by the radiation treatment machine, the time during which the radiation treatment machine has been used, the type of radiation used by the radiation treatment machine, and information about a technical staff in charge.

The management system may further include a reference value setting unit, within the processor, configured to collect statistics of occurrences of erroneous operation in the radiation treatment machine based on a total amount of radiation that has been emitted by the radiation treatment machine and a total number of times that the radiation treatment machine has emitted radiation and to set the predetermined reference value.

The management unit may send a report to an administrator that the cumulative amount of use of the radiation treatment machine will exceed the predetermined reference value if, as a result of the check, it is determined that the cumulative amount of use of the radiation treatment machine has exceeded the predetermined reference value.

The information about the treatment plan may include at least one of the time at which treatment is to be performed, a body part to be treated, the type of radiation to be used, a location at which radiation is to be emitted, the ID information of a radiation treatment machine to be used, the amount of radiation to be emitted, and the number of times that radiation is to be emitted.

In accordance with another aspect of the present invention, there is provided a management system for managing a radiation treatment machine, including a storage device and a processor. The processor may include an information acquisition unit configured to acquire information about a result of treatment of the radiation treatment machine. The storage device may include a treatment result database configured to store the information about the result of treatment. The processor may include a management unit configured to determine whether the radiation treatment machine is normal or not by analyzing results of treatment of patients, who have been exposed to the radiation treatment machine, stored in the treatment result database in order to prevent a failure and erroneous operation of the radiation treatment machine.

The management unit may report that abnormality has been found in the radiation treatment machine if, as a result of the determination, it is determined that abnormality is present in the radiation treatment machine.

The management unit may output the cumulative amount of use of the radiation treatment machine in a graph form.

In accordance with another aspect of the present invention, there is provided a method of managing a radiation treatment machine, including acquiring information about the amount of use of the radiation treatment machine; storing the information about the amount of use of the radiation treatment machine in an amount-of-use database; acquiring information about a treatment plan; storing the time at which treatment is to be performed and information about the amount of use of the radiation treatment machine when the treatment is to be performed based on the information about the treatment plan in a treatment plan database; checking the cumulative amount of use of the radiation treatment machine when the treatment is performed based on the information about the amount of use of the radiation treatment machine, which is stored in the amount-of-use database, and the time at which the treatment is to be performed and the information about the amount of use of the radiation treatment machine when the treatment is to be performed, which are stored in the treatment plan database; and checking whether the cumulative amount of use of the radiation treatment machine will exceed a predetermined reference value in order to prevent a failure and erroneous operation of the radiation treatment machine.

The information about the amount of use of the radiation treatment machine may include at least one of the amount of radiation of the radiation treatment machine, the number of times that radiation has been emitted by the radiation treatment machine, the time during which the radiation treatment machine has been used, the type of radiation used by the radiation treatment machine, and information about a technical staff in charge.

The method may further include sending a report to an administrator that the cumulative amount of use of the radiation treatment machine will exceed the predetermined reference value if, as a result of the check, it is determined that the cumulative amount of use of the radiation treatment machine has exceeded the predetermined reference value.

The information about the treatment plan may include at least one of the time at which treatment is to be performed, a body part to be treated, the type of radiation to be used, a location at which radiation is to be emitted, the ID information of a radiation treatment machine to be used, the amount of radiation to be emitted, and the number of times that radiation is to be emitted.

In accordance with another aspect of the present invention, there is provided a method of managing a radiation treatment machine, including acquiring information about a result of treatment of the radiation treatment machine; storing the information about the result of treatment in a treatment result database; and determining whether the radiation treatment machine is normal or not by analyzing treatment results of patients, who have been exposed to the radiation treatment machine, stored in the treatment result database in order to prevent the failure and erroneous operation of the radiation treatment machine.

The method may further include sending a report that abnormality has been found in the radiation treatment machine if, as a result of the determination, it is determined that abnormality is present in the radiation treatment machine.

### BRIEF DESCRIPTION OF THE DRAWINGS

The above and other objects, features and advantages of the present invention will be more clearly understood from the following detailed description taken in conjunction with the accompanying drawings, in which:

FIG. 1 schematically illustrates a typical system for managing a radiation treatment machine;

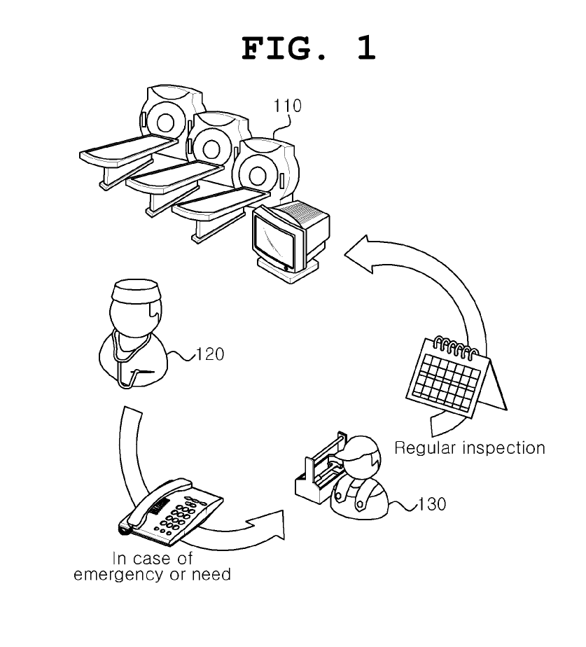

FIG. 2 schematically illustrates a system for managing a radiation treatment machine according to an embodiment of the present invention;

FIG. 3 illustrates a management system for managing a radiation treatment machine according to an embodiment of the present invention;

FIG. 4 illustrates a flowchart illustrating the management of the radiation treatment machine based on the amount of use according to an embodiment of the present invention;

FIG. 5 illustrates a flowchart illustrating the management of the radiation treatment machine based on a treatment plan according to an embodiment of the present invention;

FIG. 6 illustrates a flowchart illustrating the determination of whether the radiation treatment machine is normal or not based on the result of treatment according to an embodiment of the present invention;

FIG. 7 illustrates an example in which a message is output when the amount of use of the radiation treatment machine has exceeded a reference value according to an embodiment of the present invention;

FIG. 8 illustrates an example in which a screen used to manage the radiation treatment machine is output according to an embodiment of the present invention;

FIG. 9 is a graph illustrating the cumulative amount of use of the radiation treatment machine according to an embodiment of the present invention;

FIG. 10 illustrates a screen in which the inspection of the radiation treatment machine is requested according to an embodiment of the present invention; and

FIG. 11 illustrates a screen in which the management of a radiation treatment machine is configured according to an embodiment of the present invention.

### DETAILED DESCRIPTION OF THE DISCLOSURE

Embodiments of the present invention will be described in detail with reference to the accompanying drawings. In the following description of the present invention, detailed descriptions of related well-known components or functions that may unnecessarily make the gist of the present invention obscure will be omitted.

The prevent invention is not limited to the embodiments. Throughout the accompanying drawings, the same reference symbols are assigned to the same components.

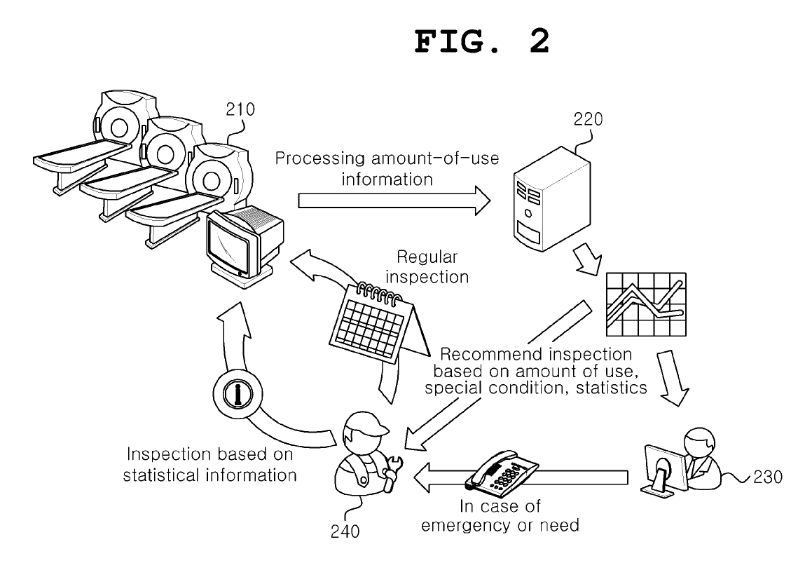

Hereinafter, a system and method for managing a radiation treatment machine (or a radiation treatment apparatus) according to embodiments of the present invention are described in detail with reference to FIGS. 2 to 11.

FIG. 2 schematically illustrates a system for managing a radiation treatment machine according to an embodiment of the present invention.

Referring to FIG. 2, a management system 220 receives information about the amount of use from a radiation treatment machine 210, checks whether the amount of use has exceeded a reference value, and sends a report to an administrator 230 or an administration engineer 240 that the radiation treatment machine 210 needs an inspection if, as a result of the check, it is determined that the amount of use has exceeded the reference value.

The administrator 230 requests the administration engineer 130 to inspect the radiation treatment machine 210 when he or she receives a report that the radiation treatment machine 210 needs an inspection from the management system 220 or when abnormality occurs in the radiation treatment machine 210.

The administration engineer 240 inspects the radiation treatment machine 210 when a report that the radiation treatment machine 210 needs an inspection is received from the management system 220, a repair or inspection request is received from the administrator 230, or a regular inspection date has been reached.

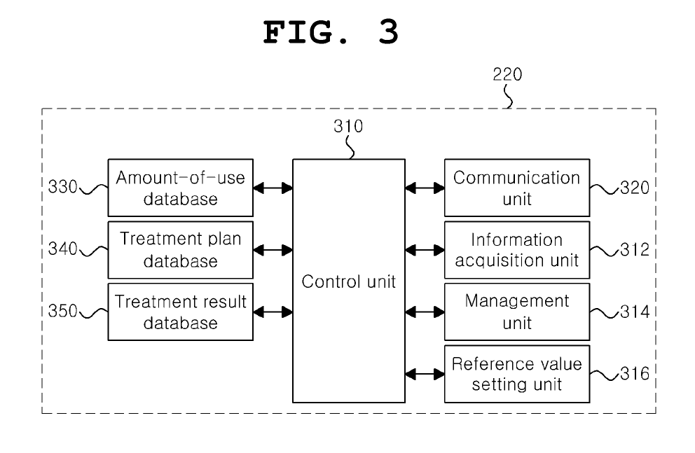

The management system 220 for managing the radiation treatment machine 210 according to an embodiment of the present invention is described in detail below with reference to FIG. 3.

FIG. 3 illustrates the management system 220 for managing the radiation treatment machine 210 according to the present embodiment.

Referring to FIG. 3, the management system 220 for managing the radiation treatment machine 210 may include a storage device (not shown), and a processor (not shown). The processor may include a control unit 310, an information acquisition unit 312, a management unit 314, a reference value setting unit 316, a communication unit 320. The storage device may include an amount-of-use database 330, a treatment plan database 340, and a treatment result database 350.

The communication unit 320 receives information about the amount of use, information about a treatment plan, or information about the result of treatment through wired/wireless communication with a system for managing medical information, such as a radiation treatment planning (RTP) system, a radiation treatment machine and a radiation oncology information system, and provides the information to the information acquisition unit 312.

The amount-of-use database 330 stores information about the amount of use of the radiation treatment machine 210. In this case, the information about the amount of use of the radiation treatment machine 210 may include at least one of the amount of radiation of the radiation treatment machine 210, the number of times that the radiation treatment machine 210 has emitted radiation, the time during which the radiation treatment machine 210 has been used, the type of radiation used by the radiation treatment machine 210, and information about a technical staff in charge.

The treatment plan database 340 stores information about the time at which treatment is to be performed and the amount of use of the radiation treatment machine 210 when treatment is to be performed based on information about a treatment plan. In this case, the information about a treatment plan may include at least one of the time at which treatment is to be performed, a body part to be treated, the type of radiation to be used, the location at which radiation is to be emitted, the ID information of the radiation treatment machine 210 to be used, the amount of radiation to be emitted, and the number of times that radiation is to be emitted.

The treatment result database 350 stores information about the result of treatment. In this case, the information about the result of treatment may include at least one of the ID information of the radiation treatment machine 210 used for treatment, information about a change in the state of a diseased part, information about the state of normal tissue around the diseased part, and information about a side effect that has occurred in a patient. The information about the result of treatment may further include another piece of information that can be used to evaluate whether the radiation treatment machine 210 is normal or not.

The information acquisition unit 312 acquires at least one of information about the amount of use, information about a treatment plan, and information about the result of treatment of the radiation treatment machine 210 via the communication unit 320.

The management unit 314 checks whether the cumulative amount of use of the radiation treatment machine 210 has exceeded a predetermined reference value based on information about the amount of use of the radiation treatment machine 210 that is stored in the amount-of-use database 330.

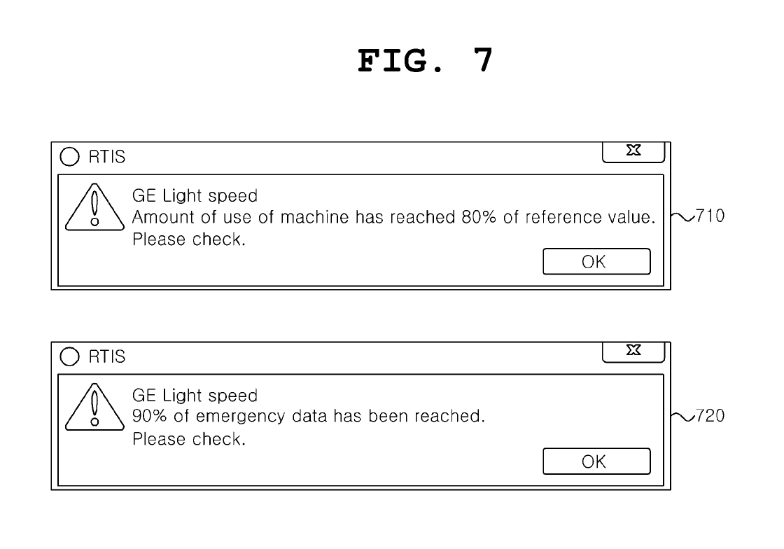

If the cumulative amount of use of the radiation treatment machine 210 has exceeded the predetermined reference value, the management unit 314 sends a report to the administrator that the cumulative amount of use of the radiation treatment machine 210 has exceeded the predetermined reference value. In this case, the predetermined reference value may be a single value, but may be set to several stepwise values, as illustrated in FIG. 7. A different reference value may be set for each type of radiation emitted by the radiation treatment machine 210.

FIG. 7 illustrates an example in which a message is output when the amount of use of the radiation treatment machine has exceeded a reference value according to an embodiment of the present invention.

Referring to FIG. 7, the management system 220 may output a corresponding report message 710 or 720 when each stepwise reference value has been exceeded.

The management unit 314 checks whether the cumulative amount of use of the radiation treatment machine 210 has exceeded a predetermined reference value when treatment is performed, using the amount-of-use database 330 and the treatment plan database 340.

If the cumulative amount of use of the radiation treatment machine 210 has exceeded a predetermined reference value, the management unit 314 sends a report to the administrator that the cumulative amount of use of the radiation treatment machine 210 has exceeded the predetermined reference value.

The management unit 314 may determine whether the radiation treatment machine 210 is normal or not by analyzing the results of the treatment of patients, who have been exposed the radiation treatment machine 210, which are stored in the treatment result database 350. In this case, the management unit 314 may determine whether the radiation treatment machine 210 is normal or not by taking into consideration information about a change in the state of a diseased part, the degree of damage to normal tissue around the diseased part, and the degree of occurrence of a side effect. Furthermore, the management unit 314 may determine whether the radiation treatment machine 210 is normal or not by comparing information about a change in the state of a diseased part, the degree of damage to normal tissue around the diseased part, and the degree of occurrence of a side effect with those of other radiation treatment machines.

If, as a result of the determination, it is determined that abnormality is present in the radiation treatment machine 210, the management unit 314 may report to the radiation treatment machine 210 that abnormality has occurred in the radiation treatment machine 210.

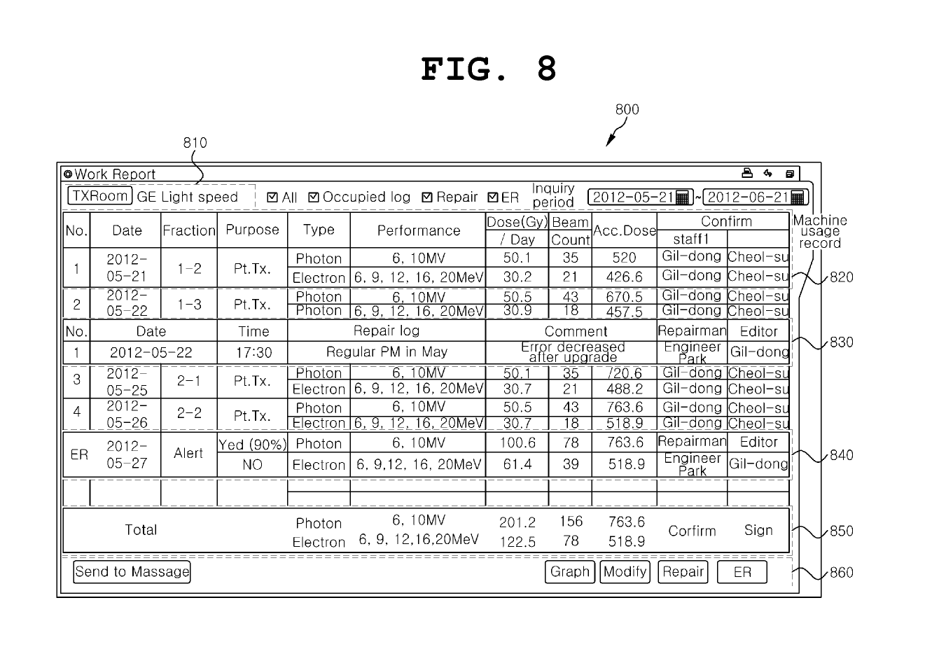

The management unit 314 may provide the administrator 230 with a management screen 800, such as that of FIG. 8.

FIG. 8 illustrates an example in which a screen used to manage the radiation treatment machine is output according to an embodiment of the present invention.

As illustrated in FIG. 8, the management unit 314 may provide the management screen 800 for each radiation treatment machine. The management screen 800 may include radiation treatment machine ID information 810, amount-of-use information 820, inspection information 830, erroneous operation information 840, cumulative amount-of-use information 850, and interface buttons 860.

The amount-of-use information 820 may include the time during which the radiation treatment machine 210 has been used, the amount of radiation emitted, fraction information, the purpose of the emission of radiation, the number of times that radiation has been emitted, the type of radiation used by the radiation treatment machine 210, and information about a technical staff in charge.

The cumulative amount-of-use information 850 may include the cumulative amount of radiation emitted and the number of times that radiation has been cumulatively emitted, with respect to each type of radiation. In this case, the cumulative amount of radiation and the number of times that radiation has been cumulatively emitted may be managed so that they are reset for each period. In this case, the period may be a predetermined time interval or may be an interval based on an inspection date.

The inspection information 830 may include information about an inspection date and repair.

The erroneous operation information 840 is information about an irregular erroneous operation, and may include information about the time at which an erroneous operation occurred and information about the processing of the erroneous operation.

The interface buttons 860 may include a report message sending button, a graph output button, a management setting button, an inspection information input button, and an error information input button.

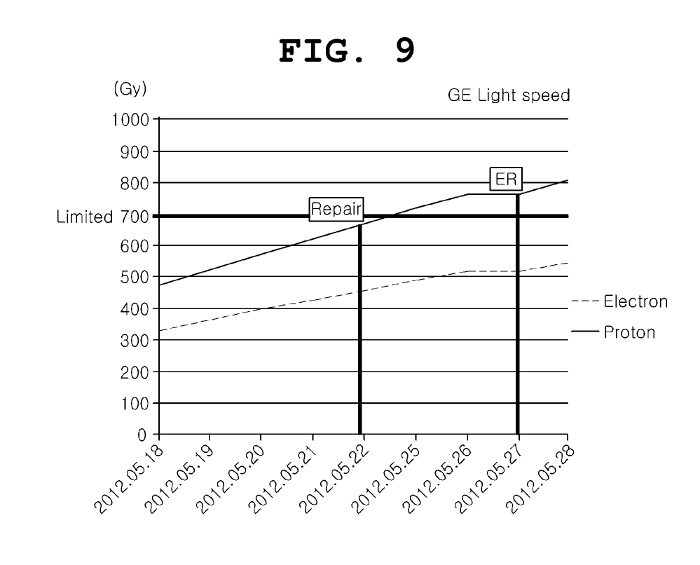

The management unit 314 may provide the administrator 230 or the administration engineer 240 with the cumulative amount of use of the radiation treatment machine 210 in a graph form, as illustrated in FIG. 9.

FIG. 9 is a graph illustrating the cumulative amount of use of the radiation treatment machine according to an embodiment of the present invention.

As illustrated in FIG. 9, the management unit 314 may display the amount of use of the radiation treatment machine 210 with respect to each type of radiation in such a way that it is accumulated over time. Furthermore, the management unit 314 may additionally display the time at which the radiation treatment machine 210 was repaired and the time at which an erroneous operation occurred in the radiation treatment machine 210 in a graph.

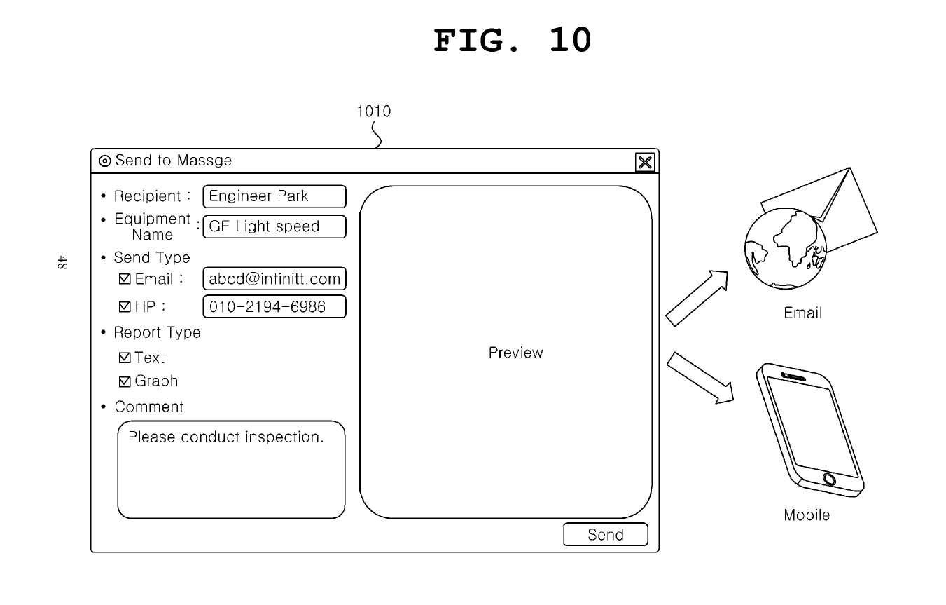

The management unit 314 may provide the administrator 230 with an interface 1010 used to request the inspection of the radiation treatment machine 210, as illustrated in FIG. 10.

FIG. 10 illustrates a screen in which the inspection of the radiation treatment machine is requested according to an embodiment of the present invention.

As illustrated in FIG. 10, the administrator 230 may request the administration engineer 240 in charge of the radiation treatment machine 210 to inspect the radiation treatment machine 210 while providing a message by using the interface 1010 provided by the management unit 314.

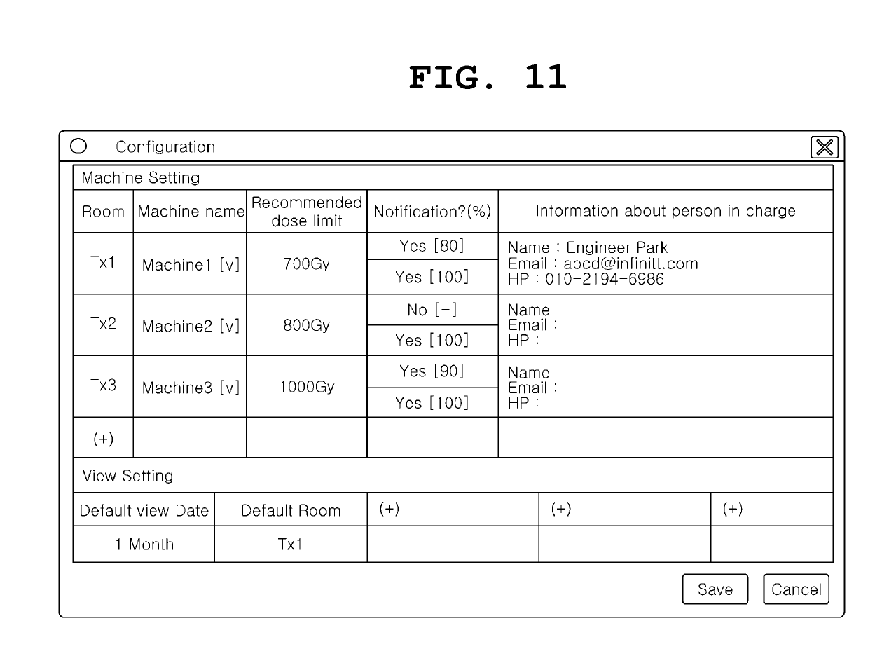

The management unit 314 may provide the administrator 230 with an interface used to manage and configure the radiation treatment machine 210, as illustrated in FIG. 11.

FIG. 11 illustrates a screen in which the management of a radiation treatment machine is configured according to an embodiment of the present invention.

As illustrated in FIG. 11, the management unit 314 may enable the administrator 230 to set a person in charge of the radiation treatment machine 210 and a reference value for an inspection report via an interface used to manage and configure the radiation treatment machine 210.

The reference value setting unit 316 may collect the statistics of the occurrences of erroneous operation in the radiation treatment machine 210 based on the cumulative amount of radiation emitted by the radiation treatment machine 210 and the number of times that radiation has been accumulated, and then may set a reference value. The reference value setting unit 316 may integrate information about the occurrence of erroneous operation in several radiation treatment machines, and may set a predetermined reference value based on statistical information.

The control unit 310 may control the overall operation of the management system 220. Furthermore, the control unit 310 may perform the functions of the information acquisition unit 312, the management unit 314, and the reference value setting unit 316. The control unit 310, the information acquisition unit 312, the management unit 314, and the reference value setting unit 316 are illustrated as being separate in order to separately describe the individual functions thereof. Accordingly, the control unit 310 may include at least one processor configured to perform each of the functions of the information acquisition unit 312, the management unit 314, and the reference value setting unit 316. Furthermore, the control unit 310 may include at least one processor configured to perform some of the functions of the information acquisition unit 312, the management unit 314, and the reference value setting unit 316.

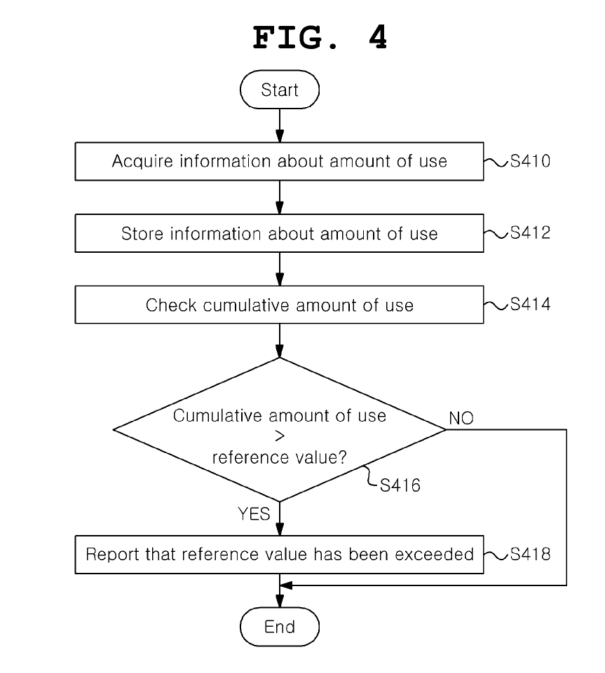

A method of managing the radiation treatment machine configured as described above according to an embodiment of the present invention is described below with reference to FIGS. 4 to 6.

FIG. 4 illustrates a flowchart illustrating the management of the radiation treatment machine based on the amount of use according to an embodiment of the present invention.

Referring to FIG. 4, in the method of managing the radiation treatment machine in the management system, information about the amount of use of the radiation treatment machine is acquired at step S410.

In this case, the information about the amount of use of the radiation treatment machine may include at least one of the amount of radiation of the radiation treatment machine, the number of times that the radiation treatment machine has emitted radiation, and the type of radiation used by the radiation treatment machine.

In the method of managing the radiation treatment machine, information about the amount of use of the radiation treatment machine acquired at step S41 is stored in the amount-of-use database at step S412.

Furthermore, in the method of managing the radiation treatment machine, the management system checks the cumulative amount of use of the radiation treatment machine based on the information about the amount of use of the radiation treatment machine that is stored in the amount-of-use database at step S414.

Furthermore, in the method of managing the radiation treatment machine, the management system checks whether the cumulative amount of use of the radiation treatment machine has exceeded a predetermined reference value at step S416. In this case, the predetermined reference value may be a single value, but may be stepwise reference values. A different reference value may be set for each type of radiation.

In the method of managing the radiation treatment machine, if, as a result of the check at step S416, it is determined that the cumulative amount of use of the radiation treatment machine has exceeded the predetermined reference value, the management system notifies the administrator that the cumulative amount of use of the radiation treatment machine has exceeded the predetermined reference value at step S418.

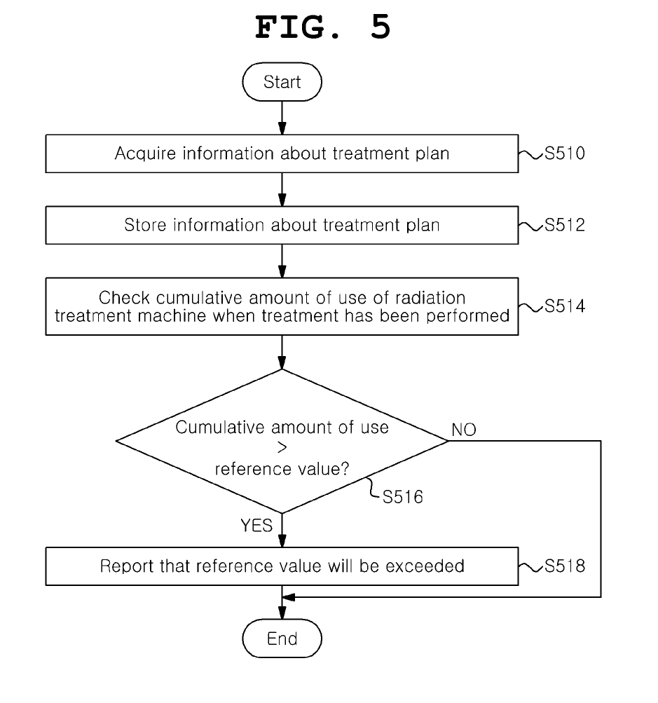

FIG. 5 illustrates a flowchart illustrating the management of the radiation treatment machine based on a treatment plan according to an embodiment of the present invention.

Referring to FIG. 5, in the method of managing the radiation treatment machine in the management system, information about a treatment plan is acquired at step S510.

In this case, the information about a treatment plan may include at least one of the time at which treatment is to be performed, a body part to be treated, the type of radiation to be used, the location at which radiation is to be emitted, the ID information of the radiation treatment machine to be used, the amount of radiation to be emitted, and the number of times that radiation is to be emitted.

In the method of managing the radiation treatment machine, the time at which treatment is to be performed and information about the amount of use of the radiation treatment machine when treatment is to be performed based on the information about the treatment plan are stored in the treatment plan database at step S512.

Furthermore, in the method of managing the radiation treatment machine, the cumulative amount of use of the radiation treatment machine when treatment is performed is checked using the amount-of-use database and the treatment plan database at step S514.

Thereafter, in the method of managing the radiation treatment machine, whether the cumulative amount of use has exceeded a predetermined reference value is checked at step S516. In this case, the predetermined reference value may be a single value, but may be stepwise reference values. A different reference value may be set for each type of radiation.

In the method of managing the radiation treatment machine, if, as a result of the check at step S516, it is determined that the cumulative amount of use of the radiation treatment machine will exceed the predetermined reference value, the administrator is notified that the cumulative amount of use of the radiation treatment machine will exceed the predetermined reference value at step S518.

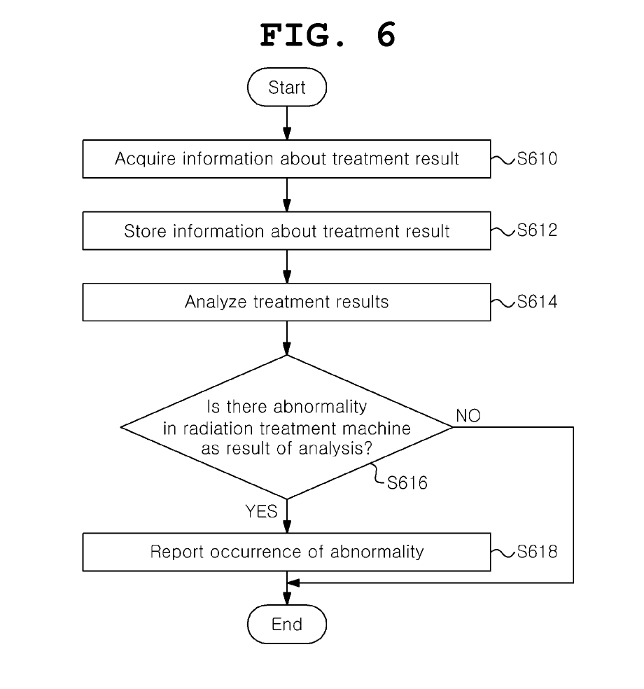

FIG. 6 illustrates a flowchart illustrating the determination of whether the radiation treatment machine is normal or not based on the result of treatment according to an embodiment of the present invention.

Referring to FIG. 6, in the method of managing the radiation treatment machine in the management system, information about the result of treatment is acquired at step S610.

Furthermore, in the method of managing the radiation treatment machine, information about the result of treatment is stored at step S612. In this case, the information about the result of treatment may include at least one of the ID information of the radiation treatment machine used for treatment, information about a change in the state of a diseased part, information about the state of normal tissue around the diseased part, and information about a side effect that has occurred in a patient.

Thereafter, in the method of managing the radiation treatment machine, the stored results of treatment of patients who have been exposed the radiation treatment machine are analyzed at step S614.

In the method of managing the radiation treatment machine, whether the radiation treatment machine is normal or not is determined based on the result of the analysis at step S616.

In this case, whether the radiation treatment machine is normal or not may be determined by taking into consideration information about a change in the state of a diseased part, the degree of damage to normal tissue around the diseased part, and the degree of occurrence of a side effect with respect to a patient who has used the corresponding radiation treatment machine, or may be determined through the comparison with the results of the treatment of other radiation treatment machines.

In the method of managing the radiation treatment machine, if, as a result of the determination at step S616, it is determined that abnormality is present in the radiation treatment machine, it is reported that abnormality has been found in the radiation treatment machine at step S618.

The present invention relates to the management system and method for acquiring at least one of information about the amount of use of a radiation treatment machine and information about a treatment plan, checking whether the amount of use of the radiation treatment machine has exceeded a reference value, and sending a report that the radiation treatment machine needs an inspection. Accordingly, the present invention contributes to the provision of improved service to a patient by presenting a criterion for the inspection of a radiation treatment machine and preventing the erroneous operation and failure of a radiation treatment machine.

Furthermore, the present invention provides the management system and method for acquiring information about the result of treatment, determining whether the radiation treatment machine is normal or not by analyzing the results of the treatment of patients who have been exposed a radiation treatment machine, and sending a report that abnormality has been found in the radiation treatment machine if, as a result of the determination, it is determined that abnormality is present in the radiation treatment machine. Accordingly, the erroneous operation and failure of the radiation treatment machine can be found.

The system and method for managing a radiation treatment machine in accordance with embodiments of the present invention may be implemented in the form of program instructions that can be executed by a variety of computer means, and may be stored in a computer-readable storage medium. The computer-readable storage medium may include program instructions, a data file, and a data structure solely or in combination. The program instructions that are stored in the medium may be designed and constructed particularly for the present invention, or may be known and available to those skilled in the field of computer software. Examples of the computer-readable storage medium include magnetic media such as a hard disk, a floppy disk and a magnetic tape, optical media such as CD-ROM and a DVD, magneto-optical media such as a floptical disk, and hardware devices particularly configured to store and execute program instructions such as ROM, RAM, and flash memory. Examples of the program instructions include not only machine language code that is constructed by a compiler but also high-level language code that can be executed by a computer using an interpreter or the like. The above-described hardware components may be configured to act as one or more software modules that perform the operation of the present invention, and vice versa.

The present invention has been derived from research conducted as part of Knowledge Economy Technology Innovation Project (Global Advanced Technology Development Project (WC-300)) of the Korean Ministry of Knowledge Economy (MKE) and the Korean Evaluation Institute of Industrial Technology [Project Management Number: 10040362; Project Name: Development of Integrated Radiation Treatment Management Solution].

While the present invention has been described in conjunction with specific details, such as specific elements, and limited embodiments and diagrams above, these are provided merely to help an overall understanding of the present invention. The present invention is not limited to these embodiments, and various modifications and variations can be made based on the foregoing description by those having ordinary knowledge in the art to which the present invention pertains.

Accordingly, the technical spirit of the present invention should not be determined based on only the described embodiments, and the following claims, all equivalents to the claims and equivalent modifications should be construed as falling within the scope of the spirit of the present invention.

What is claimed is:

1. A management system for managing a radiation treatment machine, comprising:
a storage device configured to:
store an amount-of-use database stores the information about an amount of use of the radiation treatment machine; and
store a treatment plan database stores a time at which treatment is to be performed and information about an amount of use of the radiation treatment machine when the treatment is to be performed based on information about a treatment plan; and
a processor configured to:
acquire information about the amount of use of the radiation treatment machine and the information about the treatment plan; and
check whether a cumulative amount of use of the radiation treatment machine will exceed a predetermined reference value when the treatment is to be performed, based on the information about the amount of use of the radiation treatment machine, which is stored in the amount-of-use database, and the time at which the treatment is to be performed and the information about the amount of use of the radiation treatment machine when the treatment is to be performed, which are stored in the treatment plan database, in order to prevent a failure and erroneous operation of the radiation treatment machine.

2. The management system of claim 1, wherein the information about the amount of use of the radiation treatment machine comprises at least one of an amount of radiation of the radiation treatment machine, a number of times that radiation has been emitted by the radiation treatment machine, a time during which the radiation treatment machine has been used, a type of radiation used by the radiation treatment machine, and information about a technical staff in charge.

3. The management system of claim 1, the processor is further configured to:
collect statistics of occurrences of erroneous operation in the radiation treatment machine based on a total amount of radiation that has been emitted by the radiation treatment machine and a total number of times that the radiation treatment machine has emitted radiation; and
set the predetermined reference value.

4. The management system of claim 1, wherein the processor is further configured to send a report to an administrator that the cumulative amount of use of the radiation treatment machine will exceed the predetermined reference value if, as a result of the check, it is determined that the cumulative amount of use of the radiation treatment machine has exceeded the predetermined reference value.

5. The management system of claim 1, wherein the information about the treatment plan comprises at least one of a time at which treatment is to be performed, a body part to be treated, a type of radiation to be used, a location at which radiation is to be emitted, ID information of a radiation treatment machine to be used, an amount of radiation to be emitted, and a number of times that radiation is to be emitted.

6. A management system for managing a radiation treatment machine, comprising:
a storage device configured to store a treatment result database stores information about a result of treatment; and
a processor configured to:
acquire the information about the result of treatment of the radiation treatment machine; and
determine whether the radiation treatment machine is normal or not by analyzing results of treatment of patients, who have been exposed to the radiation treatment machine, stored in the treatment result database in order to prevent a failure and erroneous operation of the radiation treatment machine.

7. The management system of claim 6, wherein the processor is further configured to send a report that abnormality has been found in the radiation treatment machine if, as a result of the determination, it is determined that abnormality is present in the radiation treatment machine.

8. The management system of claim 1, wherein the processor is further configured to output a cumulative amount of use of the radiation treatment machine in a graph form.

9. A method of managing a radiation treatment machine, comprising:
acquiring, by a processor, information about an amount of use of the radiation treatment machine;
storing, at a storage device, the information about the amount of use of the radiation treatment machine in an amount-of-use database;
acquiring, by the processor, information about a treatment plan;
storing, at the storage device, a time at which treatment is to be performed and information about an amount of use of the radiation treatment machine when the treatment is to be performed based on the information about the treatment plan in a treatment plan database;
checking, by the processor, a cumulative amount of use of the radiation treatment machine when the treatment is performed based on the information about the amount of use of the radiation treatment machine, which is stored in the amount-of-use database, and the time at which the treatment is to be performed and the information about the amount of use of the radiation treatment machine when the treatment is to be performed, which are stored in the treatment plan database; and
checking, by the processor, whether the cumulative amount of use of the radiation treatment machine will exceed a predetermined reference value in order to prevent a failure and erroneous operation of the radiation treatment machine.

10. The method of claim 9, wherein the information about the amount of use of the radiation treatment machine comprises at least one of an amount of radiation of the radiation treatment machine, a number of times that radiation has been emitted by the radiation treatment machine, a time during which the radiation treatment machine has been used, a type of radiation used by the radiation treatment machine, and information about a technical staff in charge.

11. The method of claim 9, further comprising:
sending, by the processor, a report to an administrator that the cumulative amount of use of the radiation treatment machine will exceed the predetermined reference value if, as a result of the check, it is determined that the cumulative amount of use of the radiation treatment machine has exceeded the predetermined reference value.

12. The method of claim 9, wherein the information about the treatment plan comprises at least one of a time at which treatment is to be performed, a body part to be treated, a type of radiation to be used, a location at which radiation is to be emitted, ID information of a radiation treatment machine to be used, an amount of radiation to be emitted, and a number of times that radiation is to be emitted.

13. A method of managing a radiation treatment machine, comprising:
acquiring, by a processor, information about a result of treatment of the radiation treatment machine;
storing, at a storage device, the information about the result of treatment in a treatment result database; and
determining, by the processor, whether the radiation treatment machine is normal or not by analyzing treatment results of patients, who have been exposed to the radiation treatment machine, stored in the treatment result database in order to prevent a failure and erroneous operation of the radiation treatment machine.

14. The method of claim 13, further comprising:
sending, by the processor, a report that abnormality has been found in the radiation treatment machine if, as a result of the determination, it is determined that abnormality is present in the radiation treatment machine.
